(19) United States
(12) Patent Application Publication
(10) Pub. No.: US 2022/0313483 A1
(43) Pub. Date: Oct. 6, 2022

(54) DEVICE FOR IN-SITU COOLING OF BODY-INTERNAL BIOLOGICAL TISSUES

(71) Applicants: Stefan TODOROV, Bayreuth (DE); Wilfried ERB, Erlenmoos (DE)

(72) Inventors: Stefan TODOROV, Bayreuth (DE); Wilfried ERB, Erlenmoos (DE)

(21) Appl. No.: 17/613,154

(22) PCT Filed: May 13, 2020
(86) PCT No.: PCT/EP2020/063293

§ 371 Date: Nov. 22, 2021

(30) Foreign Application Priority Data
May 22, 2019 (DE) 10 2019 207 509.6

(51) Int. Cl. A61F 7/12; A61F 7/00

(52) U.S. Cl. CPC A61F 7/12; A61F 7/0085; A61F 2007/126; A61F 2007/005; A61F 2007/0056; A61F 2007/0095

(57) ABSTRACT

A method is provided for in-situ cooling of biological tissue within the body, the biological tissue being selected from the group consisting of organ tissue, blood vessel tissue and combinations thereof. The method comprises establishing a heat-conducting contact between a heat absorption zone of a cooling unit of a device for in-situ cooling of biological tissue and the biological tissue within the body, transporting thermal energy from the heat absorption zone of the cooling unit via a substance for transporting thermal energy, which is arranged within a hollow tube of the device for in-situ cooling of biological tissue, to a cooling device of the device for in-situ cooling of biological tissue, and releasing the thermal energy via the cooling device. The method allows protecting biological tissue within the body in a location-selective manner from cancer-therapy-caused damage during cancer therapy in a simple, cost-effective and low-risk manner.

A device is provided for in-situ cooling of biological tissue within the body. The device comprises a cooling device and an implantable and sterile cooling unit having a heat absorption zone suitable for establishing heat-conducting contact with tissue within the body. Furthermore, the device has at least one hollow tube comprising or consisting of plastic and connecting the cooling device to the cooling unit, and comprising a substance for transporting thermal energy, which substance is arranged inside the hollow tube. The device is therefore suitable for transferring thermal energy to the material for transporting thermal energy at the heat absorption zone of the cooling unit and for transporting it to the cooling device. Using the device, it is possible to protect biological tissue within the body in a location-selective manner from cancer-therapy-caused damage during cancer therapy in a simpler, more cost-effective and low-risk manner.

It is known that the likelihood of pregnancy after cancer treatment can be significantly reduced (for example, by around 50%). In chemotherapy and/or radiotherapy used for cancer treatment, the risk is very high that ovarian tissue is at least partially destroyed. Even ovarian failure rates of about 92-100% are known.

The risk of developing ovarian damage (for example, ovarian atrophy and/or ovarian failure) due to cancer therapy is determined, on the one hand, by the age of the patient and, on the other hand, by the amount, type and duration of the application of a chemotherapeutic agent and/or the dose and duration of the application of radiation. Chemotherapeutic agents can lead to apoptosis and/or autophagy in growing follicles or primordial follicles of the ovarian tissue. Furthermore, chemotherapy can induce follicle activation of dormant follicles and thus lead to a “burn out” of the ovarian reserve. Radiotherapy is known to cause severe damage to the patient's DNA, wherein together with alkylating substances (such as cyclophosphamides or busulfan), a strong gonadotoxic effect has been observed not only in rapidly dividing cells but also in resting cells.

According to the recommendations of the ASCO (American Society of Clinical Oncology), the ASRM (American Society of Reproductive Medicine) and the ESHRE (European Society of Human Reproduction), all diseases that require ovarian toxic therapy are an indication for ovarian protection or the preservation of fertility. There is thus a need to protect women's ovaries from cancer treatment damage during cancer treatment.

For this purpose, it is known, for example, to fix the ovaries outside the radiation field in the case of radiotherapy, to influence the ovarian function with medication during chemotherapy and/or radiotherapy (for example, treatment with GnRH), or to preserve egg cells or ovarian tissue (for example, cortical strips or the entire organ) at a very low temperature before cancer therapy (cryopreservation).

However, these known measures are either technically difficult and associated with a certain risk (for example, cryopreservation for unfertilized egg cells and methods in which ovaries are removed from the radiation field), expensive and associated with considerable side effects (for example, drug interference) and/or ineffective in the event of chemotherapy.

In cryopreservation, which is currently the most frequently used, there is a risk that this measure will damage the follicles and that after transplantation of the cryopreserved material, it may take up to four days for new vessels to sprout. This long period of time until new vessels sprout can lead to up to 90% of originally undamaged, cryopreserved cells dying off and no longer available for fertilization.

It is previously known that cooling a person's scalp while this person is being administered a chemotherapeutic agent leads to a reduction in hair loss, the known side effect of chemotherapy (see, for example, WO 2015/082455 A1).

US 2007/0225781 A1 discloses methods and implantable devices for cooling and/or heating nerve bodies to about 15° C. in order to reduce nerve impulses.

US 2017/0348146 A1 discloses methods and implantable devices for cooling and/or heating a spinal canal and the nerve roots emanating therefrom to a temperature in a specific temperature range in order to reduce post-operative damage.

Based on this, the object of the present invention was to provide a device and a method with which it is possible to protect specific biological tissue within the body (for example, ovarian tissue of women or gonads of men) from cancer therapy-related damage during cancer therapy (chemotherapy and/or radiotherapy) in a location-selective manner. The device and the method should be suitable for enabling protection in a simpler and more cost-effective manner than known devices and methods from the prior art, while keeping the risk and side effects for the patient as low as possible.

The object is achieved by the device having the features according to claim 1, the use of the device having the features according to claim 19 and the method having the features according to claim 20. The dependent claims show advantageous embodiments.

A device for in situ cooling of biological tissue within the body selected from the group consisting of organ tissue (that is, an organ or part thereof), blood vessel tissue (that is, a blood vessel or part thereof) and combinations thereof is provided, the device comprising
i) a cooling device;
ii) an implantable and sterile cooling unit having a heat absorption zone, wherein the heat absorption zone is suitable for establishing heat-conducting contact with a tissue within the body;
iii) at least one hollow tube comprising or consisting of plastic, wherein the hollow tube connects the cooling device to the cooling unit; and
iv) a substance for transporting thermal energy, wherein the substance for transporting thermal energy is arranged within the hollow tube.

The device is therefore suitable for transferring thermal energy to the material for transporting thermal energy at the heat absorption zone of the cooling unit and for transporting it to the cooling device.

Furthermore, the device is suitable for in-situ cooling of the above-mentioned biological tissue. This means that the heat absorption zone of the device must necessarily be suitable for establishing sufficient heat-conducting contact with the tissue mentioned. This is not the case for all conceivable devices. For example, heat absorption zones of devices that are suitable for cooling the scalp or for cooling nerves necessarily have a quality (for example, shape and/or dimensioning) that makes them unsuitable for in-situ cooling of the above-mentioned, biological tissue within the body.

According to the invention, the term “implantable” is understood to mean suitability for being able to be implanted in a human or animal body, particularly to have the necessary dimensions for this purpose.

Using the device, it is possible to protect biological tissue within the body in a location-selective manner from cancer-therapy-caused damage during cancer therapy in a simpler, more cost-effective and low-risk manner. The reasons are as follows.

The cryopreservation of the tissue to be protected, which is otherwise used to protect biological tissue (for example, ovaries) in cancer therapy, is no longer necessary. This eliminates the steps of surgical removal of the tissue to be protected (for example, ovaries), of vascular anastomosis, of cryopreservation of the removed tissue and of transplantation of the tissue after cancer therapy. This saves time and money and the risk of complications for the patient is significantly reduced. If the tissue to be protected is ovaries, for example, there is no risk associated with transplantation that a transplanted ovary is at least partially destroyed within the first five days after transplantation due to insufficient blood flow. Since this risk is responsible for over 95% of the loss of primordial and preantral follicles, the protection provided by the use of the device according to the invention instead of the usual cryopreservation can significantly increase the probability of spontaneous pregnancy after cancer therapy has taken place.

A further advantage over cryopreservation is that only a single operation is necessary for a treatment using the device according to the invention. In contrast, cryopreservation requires at least two operations (first, removal of ovarian tissue and second, later replanting of ovarian tissue). In addition, the transplanted tissue is generally only active for a limited time, so that another operation (laparoscopy) with transplantation is necessary to replace tissue that has become inactive.

A further advantage of tissue protection using the device according to the invention is that after chemotherapy, there is no latency period until the tissue (for example, ovaries) begins to function. For example, in the case of cryopreservation of ovarian tissue, the latency period until the ovaries start to work is two to three months. This means that egg cells cannot be fertilized during this period of time. Here, the device according to the invention can ensure that affected women can realize their desire to have children more quickly than with the usual cryopreservation method. In addition, treatment with the device according to the invention allows a higher number of spontaneous pregnancies to be expected (see above).

Furthermore, the use of the device according to the invention for protecting biological tissue means that no (hemi-)castration of patients is necessary, which can make hormone replacement therapy superfluous and means significantly less psychological stress for affected patients.

In addition, when the device according to the invention is used, it is possible to dispense with the administration of medication to protect the biological tissue (for example, treatment with GnRH to protect the ovaries), which prevents the side effects associated with administration of medication.

The protective effect that can be provided by the device according to the invention is based on the possibility of locally selective cooling of biological tissue in the human body to a specific temperature below the usual body temperature, that is, below 37° C. Due to site-selective cooling, site-selective vasoconstriction and a site-selective increase in blood viscosity occur in the body at the site of the tissue to be protected, which prevents the chemotherapeutic agent from accumulating in the tissue to be protected, particularly from a temperature of ≤14° C. At a temperature of less than 8° C., however, blood coagulation would be activated on the endothelial cell, which is associated with a risk of microthrombosis formation of the ovarian vessels and ischemia. Significant circulatory disorders and microthrombosis would result below a temperature of 7° C. If the temperature were to be lowered even further, that is, below 4° C., cell edema would develop, including cell death through apoptosis or necrosis.

For these reasons, it is advantageous to cool the tissue to be protected by the device according to the invention to a temperature in the range from 8 to 14° C., wherein the range from 8 to 10° C. is particularly preferred. The protective effect is maximal and the side effects are minimal in this temperature range.

The protective effect achieved by cooling the biological tissue can also be explained by the fact that cellular metabolism is throttled, that is, the oxygen consumption and the enzyme activity of the cells decrease (for example, the oxygen consumption and the enzyme activity at 10° C. can be reduced to less than 15% compared to the oxygen consumption and enzyme activity at 37° C.). Chemotherapeutic agents that have a particularly toxic effect on cells that have a strong metabolism thus have a significantly less toxic effect on the cooled tissue.

In a preferred embodiment, the device comprises at least one further hollow tube comprising or consisting of plastic, wherein the at least one further hollow tube connects the cooling device to the cooling unit and the material for transporting thermal energy is also arranged within the further hollow tube. This embodiment enables the (cold) substance to circulate for transporting thermal energy from the cooling device via the (first) hollow tube to the heat absorption zone of the cooling unit and from there via the further hollow tube back to the cooling device. In the case of a solid material for transporting thermal energy (for example, a silver wire), however, circulation of the material is not necessary. Consequently, the device does not necessarily have to have a further hollow tube for the purpose of circulating the substance.

In a further, preferred embodiment, the device comprises at least one further hollow tube comprising or consisting of plastic, wherein the at least one further hollow tube is connected to a second and/or further implantable and sterile cooling unit(s), wherein the second and/or further cooling unit(s) is/are suitable for establishing one or a plurality of heat-conducting contact(s) with a tissue within the body and has a second and/or further heat absorption zone(s) and wherein the device is configured to transport thermal energy from the second and/or further heat absorption zone(s) of the second and/or further cooling unit(s) to the cooling device.

The device can comprise at least one temperature sensor, preferably at least two or more temperature sensors, wherein the temperature sensor(s) is/are preferably arranged in a region of the cooling unit(s) adjoining the heat absorption zone(s) of the cooling unit(s). The temperature sensor(s) (for example, in the form of a Peltier element) has/have the advantage that the actual temperature of the tissue within the body to be cooled can be measured and a specific preset temperature can be maintained more precisely, for example, via a communicative connection(s) to the cooling device (“feedback” regulation).

The temperature sensor(s) can be arranged in a region or regions of the second and/or further cooling unit(s) adjoining a second and/or further heat absorption zone(s) of a second and/or further cooling unit(s).

Furthermore, the temperature sensor(s) can be arranged in a region of the at least one hollow tube adjoining the cooling unit and/or a second and/or further cooling unit(s).

In addition, the temperature sensor(s) can be communicatively connected to the cooling device, preferably communicatively connected to a microcontroller of the cooling device.

In a preferred embodiment, the temperature sensor(s) is/are implantable and/or sterile.

In a preferred embodiment, the device has a coupling unit, preferably a skin port. The coupling unit can be connected reversibly or irreversibly to the at least one hollow tube. Furthermore, the coupling unit can be connected to at least one second hollow tube. In addition, the coupling unit can be connected to the cooling device. In addition, the coupling unit can be connected to the cooling unit and/or a second and/or further cooling unit(s). The coupling unit is preferably connected to at least one temperature sensor, preferably to at least two or more temperature sensors. If the coupling unit is a skin port, this has the advantage of the cooling unit being able to be reversibly connected to the cooling device in a non-invasive manner.

The coupling unit, preferably the skin port, can comprise or consist of a metal, particularly preferably a metal selected from the group consisting of titanium, stainless steel and combinations thereof. In a preferred embodiment, the coupling unit can be implanted at least in regions, preferably completely implantable, particularly preferably completely implantable subcutaneously. Ideally, the coupling unit is sterile at least in some regions, preferably completely sterile.

The device can comprise a microcontroller, wherein the microcontroller is preferably comprised in the cooling device of the device. The microcontroller can be configured to control the operation of the device. Particularly, the microcontroller is configured to regulate the cooling device such that a temperature of <37° C. prevails in the heat absorption zone of the cooling unit (optionally also a second and/or further heat absorption zone(s) of a second and/or further cooling unit(s) of the device), preferably a temperature in the range from 4° C. to 14° C., particularly preferably a temperature in the range from 6° C. to 12° C., very particularly preferably a temperature in the range from 8° C. to 10° C., particularly a temperature of 9° C. A temperature range of 4° C. to 14° C. is particularly preferred, since in this range, on the one hand, tissue damage due to overcooling is avoided and, on the other hand, adequate protection against cancer therapy-related damage is ensured.

The microcontroller can further be configured to regulate the cooling device such that the substance for transporting thermal energy to the cooling device has a specific temperature. In addition, the microcontroller can be configured to regulate the cooling device such that the substance for transporting thermal energy has a specific heat transport speed, preferably the substance has a specific flow speed. In addition, the microcontroller can be configured to regulate the cooling device such that the substance for transporting thermal energy to the cooling device until a target temperature is reached in the heat absorption zone of the cooling unit causes a cooling rate of 0.2 to 2.0 K/min, preferably 0.5 to 1.5 K/min, particularly preferably 0.8 to 1.2 K/min, in particular 1 K/min, in the heat absorption zone. These cooling rates are advantageous since, on the one hand, they are fast enough not to allow a therapy session to last too long and, on the other hand, they are sufficiently slow to prevent the patient from feeling uncomfortably cold while cooling.

In a preferred embodiment, the device comprises a heating device. The heating device preferably comprises an implantable and sterile heating unit having a heat release zone suitable for establishing a heat-conducting contact with a tissue within the body. The heating unit or the heat release zone of the heating unit can comprise or consist of a carbon net, a carbon fleece, a carbon fabric and/or a carbon film. In addition to “heating” the patient's body, the heating device allows additional heating of the tissue to be protected (target tissue) and/or the surrounding tissue that is not to be cooled. First, a more precise fine regulation of the temperature of the target tissue can take place. Second, the heating device makes it possible to prevent cooling of tissue within the body that is not intended to be cooled. This can happen, for example, by contact of the heat release zone with a blood-draining vessel and/or by contact of a surface of the heat absorption zone of the cooling unit that is turned away from the tissue to be cooled and/or faces a tissue that is not to be cooled, wherein such contact preferably takes place via a thermal insulation layer. Third, the heating device can be used to raise the temperature of the target tissue faster than using mere body heat, which is advantageous after cooling has ended, since the target tissue can be brought back to normal body temperature (37° C.) more quickly.

Furthermore, the heating device preferably has at least one hollow tube comprising or consisting of plastic, wherein the hollow tube connects the heating device to the heating unit.

Particularly, the heating device comprises a substance for transporting thermal energy from the heating device to the heat release zone of the heating unit, wherein the substance for transporting thermal energy is arranged within the hollow tube.

The substance preferably comprises or consists of a solid selected from the group consisting of metal, semi-metal, carbon and mixtures and combinations thereof. The substance particularly preferably comprises or consists of a solid selected from the group consisting of copper, silver, platinum, silicon, graphite, graphene and combinations thereof.

Particularly, the solid is arranged in the form of at least one wire within the hollow tube, preferably in the form of a plurality of wires, particularly preferably in the form of a wire mesh and/or a band of wires.

The device according to the invention is preferably configured to transport thermal energy from the heating device to the heat release zone of the heating unit.

In a preferred embodiment, the heat release zone is separated from the heat absorption zone of the cooling unit via a thermal insulation layer, wherein the insulation layer and heat release zone are preferably attached to a surface (or side) of the heat absorption zone which is opposite a tissue within the body to be cooled.

The heating device can have a heating power essentially corresponding to the cooling power of the cooling device. This can be the case if the heating device is part of the cooling device, for example, if the cooling device (or heating device) is a heat exchanger. The advantage of this variant of the device according to the invention is that thermal energy can be withdrawn from a specific tissue within the body (for example, the ovaries) via the cooling device (for example, via heat-conducting contact between the heat absorption zone of the cooling unit and blood-supplying vessels to the ovaries or directly with the ovaries) and the withdrawn thermal energy can be returned to the body downstream of the specific tissue (for example, the ovaries) within the body (for example, via heat-conducting contact between the heat release zone of the heating unit and blood-draining vessels from the ovaries). This has the effect that the specific tissue within the body (target tissue) can be cooled in a more locally selective manner and that body tissue located downstream of the blood circulation is prevented from being cooled. This embodiment is particularly energy-efficient in the case of a heat exchanger as a cooling device (or heating device).

The cooling device can comprise a gas, preferably a gas having a temperature of ≤14° C. (preferably 8 to 12° C.), wherein the gas particularly preferably comprises or consists of a gas selected from the group consisting of noble gases, N₂, N₂O, CO₂, and combinations and mixtures thereof.

Furthermore, the cooling device can comprise a liquid, preferably a liquid having a temperature of ≤14° C. (preferably 8 to 12° C.), wherein the liquid particularly preferably comprises or consists of a liquid selected from the group consisting of aqueous, isotonic saline solution, liquid N₂, liquid N₂O, liquid air, DMSO, propanediol, glycol, alcohols, and combinations and mixtures thereof.

In addition, the cooling device can comprise a solid, preferably a solid having a temperature of ≤14° C. (preferably 8 to 12° C.), wherein the solid particularly preferably comprises or consists of a solid selected from the group consisting of metal, semi-metal, non-metals (for example, graphite, graphene and/or solid CO₂) and mixtures and combinations thereof.

In addition, the cooling device can comprise an electrical Peltier element.

In a preferred embodiment, the cooling device is configured to apply a cooling power to the heat absorption zone(s) of the cooling unit(s), preferably controlled by a microcontroller of the device, which is calculated based on the formula

P [W]=3.7 [J/mL·K]·(310−x) [K]·Q [mL/sec]

wherein

x is the temperature in Kelvin to which cooling is to take place, wherein x is preferably in the range from 281 K to 285 K,

Q is the volume flow in liters of blood per second in the biological tissue to be cooled, wherein Q is preferably in the range of (60 to 100 mL/sec.) · is (blood flow volume of the biological tissue to be cooled/total blood flow volume). The volume flow is from 60 to 100 mL/sec. (for example, 80 mL/sec. in the resting pulse) for the total volume flow of blood in a human organism. This total volume flow of blood flows through the total blood flow volume of the human body. However, since only part of the tissue of the human body is to be cooled, the volume flow through this tissue is defined by the blood flow volume of this tissue in relation to the total blood flow volume. For example, if the blood flow volume of the tissue to be cooled is only 1/100 of the total blood flow volume, then Q is in the range of 0.01 (60 to 100 mL/sec.)=0.6 to 1.0 mL/sec. If this volume flow is to be cooled from 310 K (37° C.) to 283 K (10° C.), for example, a cooling power P of 3.7 J/mL·K·27 K·0.6 to 1.0 mL/sec.≈60 to 100 watts would be required for this. This configuration of the cooling device is associated with the advantage that the cooling power is matched to the volume flow in liters of blood per second in the biological tissue to be cooled and thus the biological tissue to be cooled can be efficiently cooled to a specific temperature, although blood that is constantly warmed up by the body flows through the blood volume flow into the biological tissue to be cooled.

The cooling device can be configured to allow its cooling power to increase linearly to a desired cooling power after starting the in-situ cooling of biological tissue within the body, preferably to increase linearly within 30 minutes to a desired cooling power (for example, from 0 watts to a value of 60 to 100 watts). This configuration is particularly preferably controlled via a microcontroller of the device, wherein the microcontroller calculates the cooling power as a function of time, particularly preferably calculates according to the formula

P [W]=3.7 [J/mLK]·(310−x)[K]·Q [mL/sec]·t [min]/30 [min]

wherein

x is the temperature in Kelvin to which cooling is to take place, wherein x is preferably in the range from 281 K to 285 K,

Q is the volume flow in liters of blood per second in the biological tissue to be cooled, wherein Q is preferably in the range of (0.05 to 0.15 mL/sec.) · is (blood flow volume of the biological tissue to be cooled/total blood flow volume), and

t is the time in minutes after starting the in-situ cooling of the biological tissue within the body. This configuration of the cooling device (linear increase in the cooling rate) has the advantage that the patient does not feel uncomfortably cold.

It is particularly advantageous if the cooling power remains constant after 30 minutes, that is, the desired target cooling power is reached after 30 minutes and there is no further increase in the cooling power. In the above example of a target cooling power of 60 to 100 watts, this would mean that the cooling power (applied to the cooling element(s)) increases linearly to 60 to 100 watts within 30 minutes, that is, amounts to 2 to 3.3 watts in the first minute, 4 to 6.7 watts in the second minute, 6 to 10 watts in the third minute, 12 to 20 watts in the sixth minute, 24 to 40 watts in the 12 minute, etc. This linear increase in cooling power has the decisive advantage that the patient's feeling of pain caused by cold is minimized, as the body can slowly adjust to the cooling.

The cooling device can furthermore be configured to allow its cooling power to drop linearly to a desired cooling power (for example, down to a cooling power of 0 watts) after the in-situ cooling of biological tissue within the body has been stopped, preferably to drop linearly within 30 minutes to a desired cooling power (for example, from 60 to 100 watts to only 2 to 3.3 watts). This configuration is particularly preferably controlled via a microcontroller of the device, wherein the microcontroller calculates the cooling power as a function of time, particularly preferably calculates according to the formula

P [W]=3.7 [J/mL·K]·(310−x)[K]·Q [mL/sec]·(30 [min]−t [min])/30 [min]

wherein

x is the temperature in Kelvin to which cooling is to take place, wherein x is preferably in the range from 281 K to 285 K,

Q is the volume flow in liters of blood per second in the biological tissue to be cooled, wherein Q is preferably in the range of (0.05 to 0.15 mL/sec.) · is (blood flow volume of the biological tissue to be cooled/total blood flow volume), and

t is the time in minutes after starting the in-situ cooling of the biological tissue within the body. This configuration of the cooling device (linear decrease in the cooling rate) has the advantage that the patient does not feel uncomfortably hot.

It is particularly advantageous if the cooling power is regulated to 0 watts after 30 minutes, that is, after 30 minutes, there is no longer any cooling power applied to the heat absorption zone(s) of the cooling unit(s). In the above example of a target cooling power of 60 to 100 watts, this would mean that the cooling power (applied to the cooling element(s)) falls linearly to 2 to 3.3 watts within 30 minutes, that is, amounts to 58 to 97 watts in the first minute, 56 to 93.3 watts in the second minute, 54 to 90 watts in the third minute, 48 to 80 watts in the sixth minute, 36 to 60 watts in the 12 minute, etc. This linear decrease in cooling power has the decisive advantage that the patient's feeling of pain caused by heat is minimized, as the body can slowly adjust to the heating.

The cooling unit preferably has a shape or consists of a shape which is selected from the group consisting of spiral, plate(s), dome, clip, clamp, half-shell, sleeve and combinations thereof.

Furthermore, the cooling unit can comprise the substance for transporting thermal energy, at least in some regions. The advantage here is that the cooling of the heat absorption zone is more efficient, since the distance to the material for transporting heat is minimized.

In addition, the cooling unit can have a cavity which has a wall thickness in the range from 0.1 to 15 mm, preferably 0.2 to 14 mm, particularly preferably 0.3 to 12 mm. The cooling unit can have a cavity, preferably at least one channel, in which the substance for transporting thermal energy is arranged at least in some regions.

In a preferred embodiment, the cooling unit is elastic at least in some regions, preferably in the region of the connection to the tube and/or the heat absorption zone. The advantage here is that contact with the tissue within the body is easier to make and heat-conducting contact can be established more easily and on the largest possible area on the tissue within the body, since any variability in the shape of the tissue that can occur between different patients can be compensated for.

The cooling unit can be flexible at least in some regions, preferably in the region of the connection to the tube and/or the heat absorption zone. The same advantage applies here as the one mentioned for elasticity.

Furthermore, the cooling unit can, at least in some regions, preferably in the region of the connection to the tube and/or the heat absorption zone, have an outer surface that has a surface roughness Rz according to DIN EN ISO 4287 in the range from 50 to 800 μm, preferably in the range from 70 to 600 μm, particularly preferably in the range from 100 to 400 μm, particularly in the range from 130 to 200 μm. The advantage of this embodiment is that the risk of adhesions forming on the cooling unit after the cooling unit has been implanted is minimized.

The cooling unit can also have a porous surface and/or a textured surface, at least in some regions.

Furthermore, the cooling unit can have pores at least in some regions, wherein the pores preferably have a pore size of 50 μm to 1000 μm, measured with electron microscopy, and/or an opening ratio of 0.1 to 0.7. The pores can comprise at least one antibiotic, preferably be filled or impregnated therewith. This is associated with the advantage that the risk of adhesions forming on the cooling unit after the cooling unit has been implanted is minimized.

The cooling unit can comprise or consist of a plastic, preferably a plastic, which is named in at least one document selected from the group consisting of EU Regulation 2017/745, ISO 10993, USP Class VI and/or an elastomer, preferably an elastomer selected from the group consisting of thermoplastic elastomer, silicone elastomer, polyurethane elastomer and mixtures and combinations thereof, wherein the elastomer is particularly preferably selected from the group consisting of TPO, TPV, TPU, TPC, TPS, PTA and mixtures and combinations thereof, particularly is selected from the group consisting of PP/EPDM, Desmopan® (Bayer), Hytrel® (DuPont), SBS, SEBS, SEPS, SEEPS and MBS, PEBA.

The cooling unit can comprise at least one biologically produced material, at least in some regions.

In a preferred embodiment, the heat absorption zone is designed in a shape which is selected from the group consisting of spiral, plate(s), dome, clip, clamp, half-shell, sleeve and combinations thereof. The shape can essentially correspond to the shape of the cooling unit.

The heat absorption zone can consist of a heat-conducting material having a thickness in the range from 0.01 to 1.0 mm, preferably 0.02 to 0.5 mm, particularly preferably 0.03 to 0.2 mm.

Furthermore, the heat absorption zone can have a width of 0.1 mm to 100 mm, preferably 0.2 mm to 50 mm, particularly preferably 0.5 mm to 10 mm, very particularly preferably 1 mm to 5 mm, and/or at least in some regions, a length from 2 to 100 mm, preferably 5 to 50 mm, very particularly preferably 10 to 20 mm.

In a preferred embodiment, the heat absorption zone is suitable for contacting the biological tissue on an area of 1 mm² to 500 cm², preferably on an area of 2 mm² to 50 cm², particularly preferably on an area of 5 mm² to 5 cm², very particularly preferably an area of 10 mm² to 1 cm², particularly an area of 20 mm² to 200 mm².

The heat absorption zone can have mechanical (for example, firmly bonded) contact with the substance for transporting thermal energy.

In an advantageous embodiment, the heat absorption zone is at least in some regions, preferably completely, elastic.

It is also preferred that the heat absorption zone is at least in some regions, preferably completely, flexible. The adjective “flexible” means that the heat absorption zone can be bent in a direction perpendicular to a surface that is spanned by the heat absorption zone.

The heat absorption zone can, in some regions, have an outer surface which has a surface roughness Rz according to DIN EN ISO 4287 in the range from 50 to 150 μm, preferably in the range from 70 to 600 μm, particularly preferably in the range from 100 to 400 μm, particularly in the range from 130 to 200 μm. Furthermore, the cooling unit can have pores at least in some regions, wherein the pores preferably have a pore size of 50 μm to 1000 μm, measured with electron microscopy, and/or an opening ratio of 0.1 to 0.7. The pores can comprise at least one antibiotic, preferably be filled or impregnated therewith. This is associated with the advantage that the risk of adhesions forming on the heat absorption zone after the cooling unit has been implanted is minimized.

In a preferred embodiment, the heat absorption zone is suitable for enclosing the tissue within the body at least partially, preferably at least 25%, particularly preferably at least 50%, very particularly preferably at least 75%, particularly 100%. The tissue can be a blood vessel, wherein the term enclosing means that the heat absorption zone is wrapped around the blood vessel in a direction essentially perpendicular to the extent of the blood vessel to the stated degree (for example, ≥75%).

The heat absorption zone can comprise or consist of a material selected from the group consisting of metal, semi-metal, carbon and mixtures and combinations thereof, at least in some regions. A material selected from the group consisting of platinum, gold, silicon, diamond and combinations thereof is particularly preferred, wherein the material particularly has the shape of a wire.

Furthermore, the heat absorption zone can comprise or consist of plastic at least in some regions. A plastic is preferred which is named in at least one document selected from the group consisting of EU Regulation 2017/745, ISO 10993, USP Class VI. In addition, an elastomer is preferred, preferably an elastomer selected from the group consisting of thermoplastic elastomer, silicone elastomer, polyurethane elastomer and mixtures and combinations thereof, wherein the elastomer is particularly preferably selected from the group consisting of TPO, TPV, TPU, TPC, TPS, PTA and mixtures and combinations thereof, particularly is selected from the group consisting of PP/EPDM, Desmopan® (Bayer), Hytrel® (DuPont), SBS, SEBS, SEPS, SEEPS and MBS, PEBA.

The heat absorption zone can comprise at least one biologically produced material, at least in some regions.

The heat absorption zone and/or the cooling unit can have, at least in some regions, a surface which comprises a medicament and/or a pharmaceutical product. The medicament and/or pharmaceutical product can be selected from the group consisting of pain relievers, anticoagulants, anticonvulsants, vasodilators and combinations thereof. The medicament is particularly preferably selected from the group consisting of acetylsalicylic acid, ibuprofen, heparin, PgE1, papaverine, nitroglycerine and combinations thereof. The advantage of this embodiment is that the risk of tissue perfusion damage occurring while the tissue is being cooled can be minimized.

The at least one hollow tube of the device can have a length in the range from 5 to 200 cm, preferably 10 to 150 cm, particularly preferably 15 to 100 cm, particularly 20 to 50 cm.

Furthermore, the at least one hollow tube can have, at least in some regions, a diameter of 0.1 to 10 mm, preferably 0.2 to 8 mm, particularly preferably 0.5 to 6 mm, particularly 1 to 4 mm.

In a preferred embodiment, the at least one hollow tube is elastic at least in some regions, preferably in the region of the connection to the cooling unit.

It is further preferred that the at least one hollow tube is flexible at least in some regions, preferably in the region of the connection to the cooling unit.

In a preferred embodiment, the at least one hollow tube has, at least in some regions, preferably in the region of the connection to the cooling unit, an outer surface that has a surface roughness Rz according to DIN EN ISO 4287 in the range from 50 to 150 μm, preferably in the range from 70 to 600 μm, particularly preferably in the range from 100 to 400 μm, particularly in the range from 130 to 200 μm. Furthermore, the cooling unit can have pores at least in some regions, wherein the pores preferably have a pore size of 50 μm to 1000 μm, measured with electron microscopy, and/or an opening ratio of 0.1 to 0.7. The pores can comprise at least one antibiotic, preferably be filled or impregnated therewith. These embodiments reduce the risk of adhesions forming on the at least one hollow tube after an (at least partial or regional) implantation of the at least one hollow tube.

The at least one hollow tube can have a greater wall thickness than the cooling unit. The advantage here is that the at least one hollow tube is more heat-insulating or more cold-insulating than the cooling unit and thus more locally selective cooling is effected at the location of the cooling unit.

Furthermore, the at least one hollow tube can have a layer for thermal insulation, preferably a layer comprising or consisting of a foamed plastic. This embodiment also contributes to improved thermal insulation of the interior of the hollow tube (in which the substance for transporting heat is located) to the exterior of the hollow tube (in which biological tissue that should not be cooled can be found).

The at least one hollow tube can advantageously be implanted at least in some regions and/or is sterile.

The plastic of the at least one hollow tube can be a plastic which is named in at least one document selected from the group consisting of EU Regulation 2017/745, ISO 10993, USP Class VI. In addition, the plastic can comprise or consist of an elastomer, preferably an elastomer selected from the group consisting of thermoplastic elastomer, silicone elastomer, polyurethane elastomer and mixtures and combinations thereof, wherein the elastomer is particularly preferably selected from the group consisting of TPO, TPV, TPU, TPC, TPS, PTA and mixtures and combinations thereof, particularly is selected from the group consisting of PP/EPDM, Desmopan® (Bayer), Hytrel® (DuPont), SBS, SEBS, SEPS, SEEPS and MBS, PEBA.

Furthermore, the plastic of the at least one hollow tube can comprise or consist of at least one biologically produced material, at least in some regions.

The substance for transporting thermal energy can comprise or consist of a gas, wherein the gas particularly preferably comprises or consists of a gas selected from the group consisting of noble gases, N₂, N₂O, CO₂ and combinations and mixtures thereof.

Furthermore, the substance for transporting thermal energy can comprise or consist of a liquid, wherein the liquid particularly preferably comprises or consists of a liquid selected from the group consisting of water, aqueous buffers and combinations and mixtures thereof, very particularly preferably a liquid selected from the group consisting of HAES Solution, Collins solution, University of Wisconsin solution, Breitschneider's solution, citrate solution and combinations thereof. The advantage of using these liquids is that they have been approved for intracorporeal use for decades and therefore do not represent a source of danger in the event of damage to (parts of the) device.

The device can comprise a pump (for example, a peristaltic and/or a membrane pump) with which the substance for transporting thermal energy is transported through the at least one hollow tube. The substance is preferably transported for transporting thermal energy from the heat absorption zone of the cooling unit to the cooling device, cooled there and transported back to the heat absorption zone. In other words, the substance for transporting heat is preferably circulated in the device via at least one pump. Such a circulation is not necessary, for example, if the substance used to transport heat is a solid. In this case, the thermal energy can be transported along the solid to the cooling device without the solid having to be moved (or circulated).

In addition, the substance for transporting thermal energy can comprise or consist of a solid, wherein the solid particularly preferably comprises or consists of a solid selected from the group consisting of metal, semi-metal, carbon and mixtures and combinations thereof, particularly preferably a solid selected from the group consisting of copper, silver, platinum, silicon, graphite, graphene and combinations thereof, and wherein the solid is arranged particularly in the form of a single wire, wire bundle, wire textile and/or wire band within the hollow tube.

In a preferred embodiment, the substance for transporting thermal energy is sterile. The advantage of this embodiment is that in the unlikely event of damage to an at least partially implanted hollow tube and/or an implanted cooling unit, contact of the substance with the interior of a patient's body cannot cause an infection.

The tissue is preferably selected from the group consisting of parenchymal organ, blood-supplying vessel thereof and combinations thereof, particularly preferably gonad, blood-supplying vessel thereof and combinations thereof. The tissue is very particularly preferably selected from the group consisting of ovaries, testes, blood-supplying and blood-draining vessel thereof and combinations thereof.

The device can be configured for intracorporeal cooling of body tissue and/or for generating hypothermia, preferably configured for cooling and/or generating hypothermia of gonadal tissue.

Furthermore, the device can be configured to reduce the blood flow in a body tissue, preferably configured by generating vasoconstriction.

In addition, the device can be configured to prevent pharmacokinetic accumulation of a chemotherapeutic agent in the gonadal tissue.

In addition, the device can be configured to reduce metabolic processes, particularly enzyme activities, in the gonadal tissue.

In a preferred embodiment, the device is configured to lower chemotherapeutic agent concentration in the ovary or in the testicle by lowering the temperature in the ovary or in the testicle.

Furthermore, the device can be configured to bring about a reduction in blood flow in the ovary or in the testes, which during chemotherapy leads to a reduction in damage to the vessels and organs to be protected.

The device can be configured to lower a chemotherapeutic agent concentration during chemotherapy in the ovary or in the testes by lowering the temperature.

In addition, the device can be configured to bring about a reduction in blood flow in the ovary or in the testes, so that the activity of the enzyme systems in the ovary or in the testes is downregulated and cytotoxic damage to the fertility organs during chemotherapy is reduced.

The device can be configured to change a metabolism of the gonads such that a radiobiological impairment of fertility is reduced in the context of radiation therapy.

Furthermore, the device can be configured so that the transport of thermal energy to the cooling device takes place via a skin port which serves as a mechanical and thermal connector between the cooling device and the heat absorption zone of the sterile and implantable cooling unit.

The skin port can constitute an interface between an extracorporeal part of the device and an intracorporeal part of the device.

The skin port can be configured to be attached extracorporeally to a living body and/or to be implanted intracorporeally, preferably subdermally, into a living body.

Furthermore, the skin port can have a plastic membrane, preferably a fluoropolymer membrane, which is preferably attached to containers made of metal or plastic.

In addition, the skin port can have connectors, preferably have connectors for needles, cannulas and/or braunules, wherein the connectors are particularly preferably configured to establish a connection to the cooling device and/or to the cooling unit.

The device can be configured to be attached to an infusion stand, preferably to be attached to a chemotherapy infusion stand, wherein the device is particularly preferably attached to an infusion stand.

The cooling unit(s) can be configured to be attached to two ovaries. One cooling unit can each be configured to be attached to an ovary.

Furthermore, the cooling unit can be configured to contact ovaries displaced extraperitoneally in order to eliminate pain.

The device can be designed as an operating set consisting of a cooling unit and a temperature sensor.

Furthermore, the device can have a laparoscopic instrument, preferably a cooling cuff with an awl, wherein the instrument preferably enables the cooling and measuring lines to be led out to the skin.

It is proposed to use the device according to the invention for in-situ cooling of biological tissue within the body, which is selected from the group consisting of organ tissue, blood vessel tissue and combinations thereof. The tissue is preferably selected from the group consisting of parenchymal organ, blood-supplying vessel thereof and combinations thereof, particularly preferably selected from the group consisting of gonad, blood-supplying vessel thereof and combinations thereof, very particularly preferably selected from the group consisting of ovary, testes, blood-supplying and blood-draining vessel thereof and combinations thereof.

Furthermore, a method is provided for in-situ cooling of biological tissue within the body, which is selected from the group consisting of organ tissue, blood vessel tissue and combinations thereof. The method comprises the steps
i) establishing a heat-conductive contact between a heat absorption zone of a cooling unit of a device for in-situ cooling of biological tissue and a biological tissue within the body; and
ii) transporting thermal energy from the heat absorption zone of the cooling unit to a cooling device of the device for in-situ cooling of biological tissue, wherein the thermal energy is transported via a substance for transporting thermal energy, which substance is arranged within a hollow tube of the device for in-situ cooling of biological tissue; and
iii) releasing the thermal energy via the cooling device.

The method can be characterized in that the tissue is selected from the group consisting of parenchymal organ, blood-supplying vessel thereof and combinations thereof, particularly preferably is selected from the group consisting of gonad, blood-supplying vessel thereof and combinations thereof, very particularly preferably is selected from the group consisting of ovaries, testes, blood-supplying and blood-draining vessel thereof, and combinations thereof.

The method can be designed as a therapeutic method. If cancer therapy is carried out on the body's biological tissue within the body while the method is being carried out, the method can be viewed as a therapeutic method. In this preferred embodiment of the method, the tissue is the biological tissue within the body of a patient to whom cancer therapy is applied during the method.

In the method, the heat absorption zone of the cooling unit can be applied to the biological tissue within the body from outside the body in order to establish a heat-conducting contact between the heat absorption zone and the biological tissue within the body. It is conceivable here to cool the testes by placing the heat absorption zone on the testes from the outside, or to cool the ovaries by placing the heat absorption zone on an inner surface of the vagina. In other words, no surgical intervention is necessary in this case to establish the heat-conducting contact. The method can thus be designed as a non-surgical method.

In the method, the cooling device, preferably also a part of the at least one hollow tube, can be arranged outside the biological tissue within the body of a patient. The thermal energy is then preferably released extracorporeally. The advantage here is that no tissue within the body is heated, which minimizes the risk of fibrosis and inflammation. Furthermore, the effort for carrying out the method is lower, because carrying out an intracorporeal heat dissipation involves greater effort, higher risk and is fraught with risks. In addition to the cooling device, the cooling unit can also be arranged outside the biological tissue within the body of the patient, particularly in the intravaginal space. The advantage is that the ovaries or their blood vessels can be cooled without surgical intervention.

In a preferred embodiment, the hollow tube, optionally also further components of the device (for example, parts of the at least one hollow tube), is/are guided extraperitoneally out of a patient's body by the shortest route. The advantage is that the risks of fibrosis and inflammation are largely avoided.

In a particularly preferred embodiment of the method, a temperature of 4° C. to 14° C., preferably a temperature of 6° C. to 12° C., particularly preferably a temperature of 8° C. to 10° C., is set at the heat absorption zone of the cooling unit. The advantage is that tissue damage caused by overcooling is avoided and adequate protection against cancer therapy-related damage is guaranteed. The protection results from the vasoconstriction due to the low temperature. This pharmaceutically reduces the influx of therapeutic agents and the associated stress on the gonads. It thus has the gonadoprotective effect. In addition, according to the so-called Arrhenius rule, a decrease in temperature of 10° C. more than halves the metabolic rate of the gonads. When cooling to approx. 6° C., the metabolic rate drops to approx. 16% compared to the rate at regular body temperature (approx. 37° C.). This is an additional protective effect to the chemo-onco protection, and to the radiation protection of the gonads.

It is also preferred to carry out the method using a device according to the invention. Here, an above-described configuration of the device according to the invention (for example, a microcontroller of the device according to the invention) can be carried out as a method step in the method according to the invention. For example, in the method, the cooling device can be regulated such that a temperature of <37° C. prevails in the heat absorption zone of the cooling unit, preferably a temperature in the range from 4° C. to 14° C., particularly preferably a temperature in the range from 6° C. to 12° C., very particularly preferably a temperature in the range from 8° C. to 10° C., particularly a temperature of 9° C.

The method according to the invention is characterized by at least one of the following advantages:
Temporary use of a cooling unit is possible, that is, heat-conducting contact between a heat absorption zone of the cooling unit and a biological tissue within the body of a patient can be released after chemotherapy (reversibility of the method);
High efficiency in organ preservation is provided. If, on the other hand, an ovarian transplant were necessary to protect the ovaries from therapy-related damage, either the entire ovary or ⅔ of it would be destroyed. This can be avoided by the method according to the invention;
The ovarian blood flow is maintained. Furthermore, there is no need for a technically complicated and complication-prone vascular anastomosis. A lack of ovarian tissue blood flow after thawing and transplantation, which is unnecessary by the method according to the invention, can prevent over 95% of the primordial and preantral follicles from being lost. Harmful perfusion damage and hypothermia damage can be avoided via the method according to the invention;
It can be carried out quickly, that is, there is no need to wait 14 to 21 days for the operation (“random start”);
During chemotherapy, the combination with ovarian stimulation as part of IVF/ICSI treatment and egg collection or ovarian tissue watering is conceivable;
In prepuberal girls and boys, hemicastration is not necessary in the method according to the invention in order to protect the gonads from damage caused by therapy;
In high-dose chemotherapy and/or radiotherapy, the method according to the invention can preserve the ovary;
Combinations with medicinal methods of ovarian protection, such as the administration of GnRH analogues, oral contraceptives, etc., are possible;
In contrast to multiple laparoscopies in the transplantation of thawed ovarian tissue, no or only two laparoscopic operations are necessary in the method according to the invention, depending on the application;
There is no latency to ovarian function resumption after chemotherapy. In methods from the prior art, on the other hand, in transplantation of thawed ovarian tissue, the latency period up to the start of activity is approx. 2-3 months;
A higher number of spontaneous pregnancies after the therapy can be expected with the method according to the invention in comparison with methods from the prior art. In contrast, with ovarian transplantation used in the prior art, only about 40% of pregnancies are spontaneous and 60% are after IVF/ICSI treatment;
The age limit for performing the operation can be raised from 35 to 40 years, as invasiveness is lower and patient acceptance is higher;
The method can be applied to chemotherapies with lower cytotoxicity, especially for Hodgkin's disease, breast cancer and others;
The procedure can be used for testicular cancer. This is an advantage over methods from the prior art in which frozen testicular tissue transplantation has failed;
Cooling can be done in situ or transcutaneously in testicular cancer or ovarian cancer. In other words, the heat absorbing zone of the cooling unit of the device can be applied from outside the body to the testes or to the ovaries (for example, via an inside of the vagina). The advantage is that this procedure is non-invasive and does not require surgical intervention.

The subject according to the invention is intended to be explained in more detail with the aid of the following figures and the following example, without wishing to restrict it to the specific embodiments shown here.

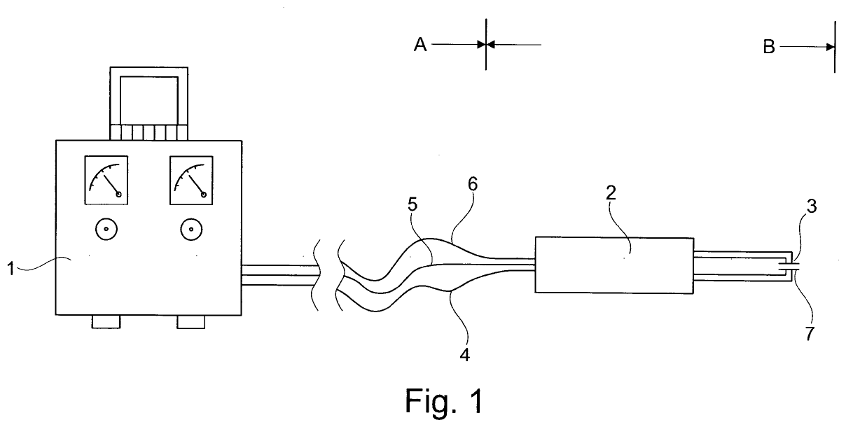

FIG. 1 shows a schematic representation of a device according to the invention. The cooling device 1 is connected to the cooling unit 2 via at least one hollow tube 4. In this embodiment, the cooling device is also connected to the cooling unit 2 via at least one further hollow tube 5 and via a communication cable 6 from a temperature sensor 7. The cooling unit 2 has a heat absorption zone 3 and a temperature sensor 7. After an operative implantation of the implantable and sterile cooling unit 2, the cooling unit 2, its heat absorption zone 3, the temperature sensor 7 and parts of the hollow tubes 4, 5 and the communication cable 6 of the temperature sensor 7 are inside the body and form an intracorporeal part B of the device. The cooling device 1 and parts of the parts of the hollow tubes 4, 5 and the communication cable 6 of the temperature sensor 7 remain outside the body and form an extracorporeal part A of the device.

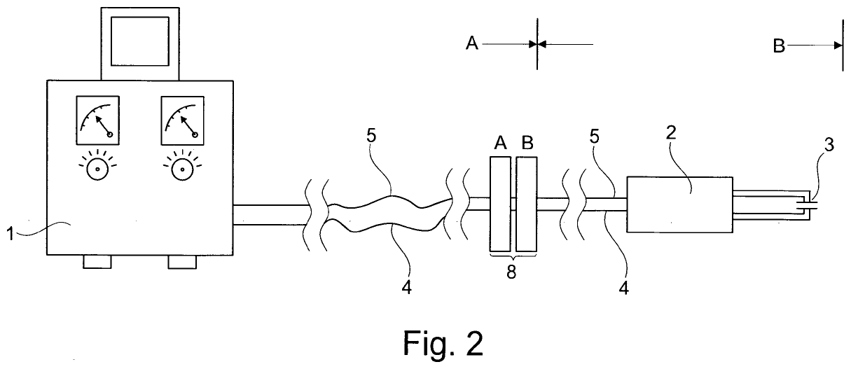

FIG. 2 shows a schematic representation of a further device according to the invention. The cooling device 1 is connected to the cooling unit 2 via at least one hollow tube 4 and via a coupling unit 8 (here: a skin port). In this embodiment, the cooling device is also connected to the cooling unit 2 via at least one further hollow tube 5 and via the coupling unit 8 (here: a skin port). The coupling unit separates the hollow tubes 4, 5 into an extracorporeal part A and an intracorporeal part B, wherein the respective parts of the tubes 4, 5 can be reversibly connected to one another on the coupling unit 8. The coupling unit is implantable and sterile and is preferably implanted on the skin or under the skin of a patient. In the case of implantation under the skin, the extracorporeal part A of the two hollow tubes must be sterile at least in some regions when connected to the coupling unit (that is, in the region that comes to lie under the skin). The cooling unit 2 has a heat absorption zone 3. After an operative implantation of the implantable and sterile cooling unit 2, the cooling unit 2, its heat absorption zone 3, parts of the hollow tubes 4, 5 and at least parts of the coupling unit 8 are inside the body and form an intracorporeal part B of the device. The cooling device land parts of the parts of the hollow tubes 4, 5 remain outside the body and optionally parts of the coupling unit form an extracorporeal part A of the device.

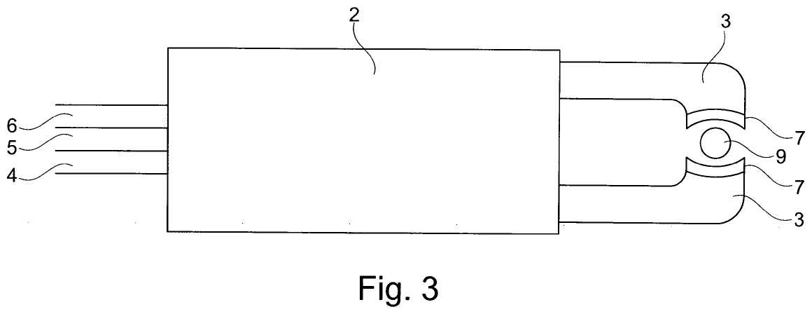

FIG. 3 shows a cooling unit 2 of a device according to the invention. The cooling unit 2 has a heat absorption zone 3 and a temperature sensor 7. The heat absorption zone 3 and the temperature sensor are suitable for establishing heat-conducting contact with tissue 9 within the body. The heat absorption zone 3 is suitable for transferring thermal energy from the tissue 9 within the body to a substance for transporting thermal energy (not shown) which is located in the hollow tube 4. Here, the substance for transporting thermal energy is liquid and after it has absorbed thermal energy at the heat absorption zone 3, it is transported via at least one further hollow tube 5 to the cooling unit (not shown), where thermal energy is withdrawn from it (that is, where it is cooled). The substance cooled by the cooling device is then transported again via the hollow tube 4 to the heat absorption zone 3 of the cooling unit 2, where it can again absorb thermal energy from the tissue 9 via the heat absorption zone 3 of the cooling unit and transport it via the at least one further hollow tube 5 to the cooling device. The temperature present on the tissue 9 is detected via the temperature sensor 7 and the detected temperature is transmitted to the cooling device via the communication cable 6. In this embodiment, the cooling device is configured to regulate the cooling power as a function of the transmitted temperature. This regulation can take place via a microcontroller of the cooling device.

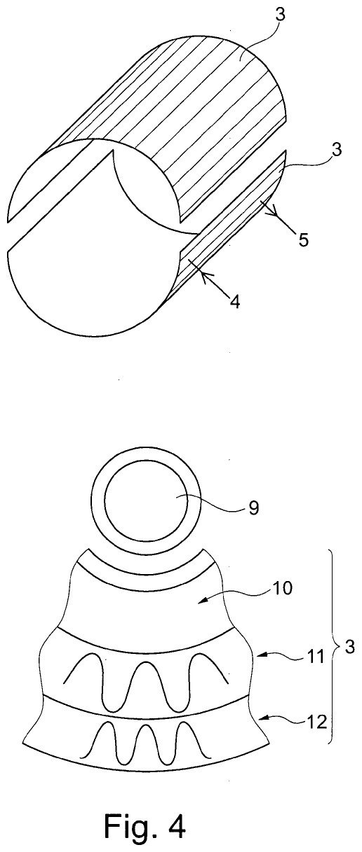

FIG. 4 shows a heat absorption zone 3 of a cooling unit of a device according to the invention. The heat absorption zone 3 is connected to a hollow tube 4 for supplying cooled substance 10 for transporting thermal energy and connected to a further hollow tube 5 for removing the heated substance 10 for transporting thermal energy. The heat absorption zone is here in the shape of a bowl and is formed over three layers lying one on top of the other. The first layer is located close to the biological tissue 9 to be cooled (here: a blood vessel leading to the ovary), is hollow, and the substance 10 flows through it for transporting thermal energy. The second layer 11 is located in a direction away from the tissue 9 adjacent to the first layer and constitutes an insulation layer 11. The third layer is located in a direction away from the tissue 9 adjacent to the second layer 11 and constitutes a heat release zone of a heating unit of a heating device of the device according to the invention, via which this layer can be heated.

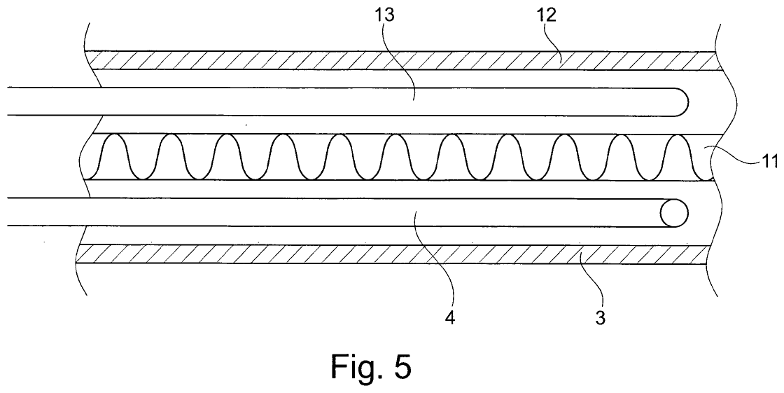

FIG. 5 shows a structure of a cooling unit of a device according to the invention. The heat absorption zone 3 of the cooling unit is arranged on a front side of the cooling unit and can transport thermal energy away via at least one substance for transporting heat, which is located in at least one hollow tube 4. The hollow tube 4 is arranged between the cooling device and the cooling unit and is also partially comprised here in the cooling unit. In this embodiment, the device according to the invention also has a heating unit which is designed as part of the cooling unit, that is, is arranged here on a rear side of the cooling unit. The heat release zone 12 of the heating unit is in thermal contact with at least one substance for transporting thermal energy, which is located in at least one hollow tube 13. The at least one hollow tube 13 is arranged between the heating device and the heating unit. In order to avoid or minimize migration of the substance in the hollow tube 13 on the rear side of the cooling unit (=heat source on the back side of the cooling unit) to the substance in the hollow tube 4 on the front side of the cooling unit (=heat sink on the front side of the cooling unit), the cooling unit has a thermal insulation layer between the two hollow tubes 4, 13. The cooling unit can have at least one temperature sensor (not shown) on the heat absorption zone 3 and/or the heat release zone 12.

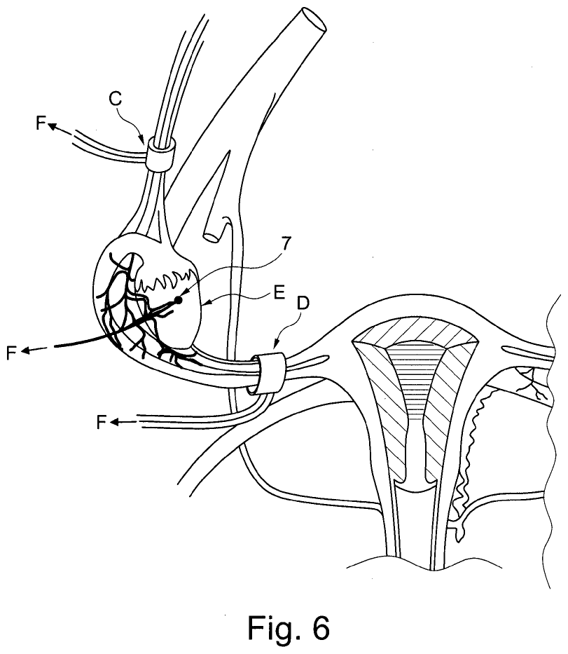

FIG. 6 shows a possible attachment of cooling units of a device according to the invention. A first cooling unit C in the form of a cooling cuff can be attached to the ligamentum suspensorium ovarii. A second cooling unit D in the form of a cooling cuff can be attached to the ligamentum ovarii proprium. A temperature sensor is attached to the ovary E to measure the temperature that prevails directly on the ovary. Lines F of the temperature sensor and the cooling units C, D lead to the cooling device (not shown) of the device according to the invention.

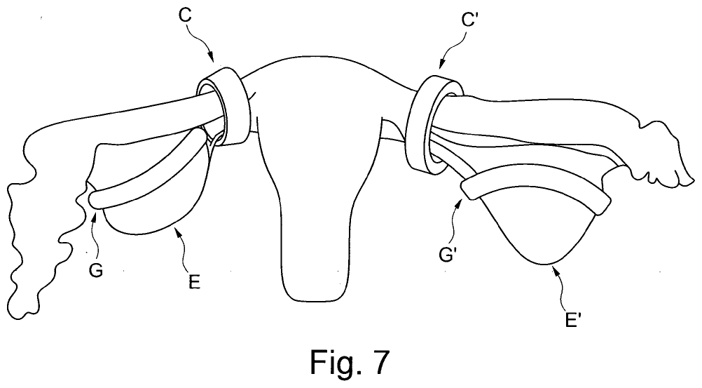

FIG. 7 shows a further possible attachment of cooling units of a device according to the invention. A first cooling unit C, C′ in the form of a cooling cuff can be attached to each of the ligamenta suspensorium ovarii. A second cooling unit G, G′ in the form of a cooling cuff can be attached to each of the ovaries E, E′. Lines of the cooling units C, C′, G, G′ lead to the cooling device (not shown) of the device according to the invention.

### EXAMPLE

### Use of a Device According to the Invention

The cooling unit of the device can be attached to the lig. cusp. ovarii or lig. ovarii proprium of a patient to be treated. If the device comprises a temperature sensor, this sensor can be attached to the mesovar of a patient to be treated. The temperature sensor enables the temperature to be monitored at the desired location and thus more precise temperature control. Cooling of the blood to 8 to 10° C. caused by the device results in rheological deprivation of the ovary and vasoconstriction of the blood vessels with the advantageous effects described.

In principle, the cooling units can have the form of cuffs or insulated clamps and, as such, can be attached to blood vessels. It is possible to attach only a small part of the circumference of the cooling cuff intraperitoneally, that is, in contact with the intestine. The advantage of the smallest possible contact area with the intraperitoneum is that a dull nerve sensation on the visceral peritoneum and the risk of forming adhesions are kept as low as possible.

The pre-cooling phase without chemotherapy takes about 30 minutes. The warm-up phase after chemotherapy is another 30 minutes. In total, a therapy session can last about 4 hours, depending on the type of chemotherapy, sometimes shorter. On average, 4 to 8 therapy sessions can be carried out at 3 to 4 week intervals. The implanted cooling units can then be removed as part of an operative laparoscopy.

Most of the hollow tube or tubes of the device are preferably deployed extraperitoneally. Direct cooling of the mesovar is conceivable, as the mesovar can easily be pulled through laparoscopically into a loop. The connection to the extraperitoneal space can be made via a peritoneal incision, which can be made transversely below the ovary. In such a case, the temperature is measured on the lig. suspensorium ovarii. Analogously, the hollow tube and part of the cuff of the device can be guided extraperitoneally. Alternatively, the cooling is realized via the lig. suspensorium ovarii, ovarii proprium and tubae uterinae. A temperature measurement can be made directly on the mesovar using a temperature sensor.

In a 2-port model or 3-port model, two skin incisions are made approx. 5 mm above the christa Iliaca anterior superior. The laparoscopic 5 mm instruments for operative endoscopy are guided intraperitoneally through the same skin incisions. The cooling lines are guided extraperitoneally through the same skin incisions. A 5 mm suprasymphyseal port is used for temperature measurement. The 10 mm incision in the umbilicus is used for intra-abdominal insertion of the cooling sets and measuring wires.

Alternatively, two operative modifications are presented below.

The peritoneal incision, which was made on the latum ligament below the ovaries, can be used extraperitoneally after the hollow tube has been laid, the ovary can be rotated about an imaginary axis frontally by 180° caudally on the mesovar by retroperitoneal transposition before the peritoneum is closed. As a result, the ovary is completely extraperitoneal, where it can be cooled, detached from the visceral pain fibers, until the end of the chemotherapy. When explanting the cooling set, the peritoneum is opened at the same point and the ovary is moved intraperitoneally on the mesovar.

A further possibility is subcutaneous epifascial transposition of the ovary at lig. infundibulopelvicum sive suspensorium ovarii. First, the uterine tuba and the lig. ovarii proprium are divided close to the uterus. Then the ovary is moved cranially and laterally to the blood supply of the lig. susp. ovarii. The fascia directly below the 5 mm port is opened to the same distance after a 2 cm skin incision, the ovary is pulled through on the vascular pedicle and placed subcutaneously epifascial. The cooling cuff can be put on here. Local anesthetic perfusion is not necessary. Upon completion of chemotherapy, the ovary is moved intraperitoneally on the pedicle.

The following approach is also possible: First, the cooling set with cuff is applied intraperitoneally through the 10 mm subumbilical port. Then peritoneal slits are made below the lig. susp. ovarii, lig. ovarii proprium and mesovar. Under laparoscopic view, an eel is introduced extraperitoneally through the 5 mm port on the side. This rail, or eel, has an eyelet at the front, which enables the cooling belt system to be threaded in. This cooling and measuring control system is passed through retrograde cutaneously and the cuffs are closed around the ovary. Care is taken to ensure that the vascular structures at the anulus internus canalis inguinalis are not injured. The cuff can be closed with a so-called “gastric banding” system.

### LIST OF REFERENCE SYMBOLS

A: extracorporeal part;

B: intracorporeal part;

C, C′: cooling unit in the form of a cooling cuff on the ligamentum suspensorium ovarii;

D: cooling unit in the form of a cooling cuff on the ligamentum ovarii proprium;

E, E′: ovary;

F: line(s) to the cooling device;

G, G′: cooling unit in the form of a cooling cuff on the ovary;

1: cooling device;

2: cooling unit;

3: heat absorption zone of the cooling unit (or part thereof);

4: at least one hollow tube between the cooling device and the cooling unit;

5: at least one further hollow tube between the cooling device and the cooling unit;

6: communication cable from temperature sensor;

7: temperature sensor;

8: coupling unit (for example, skin port);

9: tissue (to be cooled, for example, blood vessel to ovary);

10: substance for the transport of heat (for example, liquid refrigerant);

11: insulation layer;

12: heat release zone of a heating unit of a heating device.

13: at least one hollow tube between the heating device and the heating unit.

1-26. (canceled)

27. A method for in-situ cooling of a biological tissue within the body, comprising:
(i) establishing a heat-conducting contact between a heat absorption zone of a cooling unit of a device for in-situ cooling of a biological tissue and a biological tissue within the body, wherein the biological tissue is selected from the group consisting of organ tissue, blood vessel tissue and combinations thereof;
(ii) transporting thermal energy from the heat absorption zone of the cooling unit to a cooling device of the device for in-situ cooling of biological tissue, wherein the thermal energy is transported via a substance for transporting thermal energy, which substance is arranged within a hollow tube of the device for in-situ cooling of biological tissue; and
(iii) releasing the thermal energy via the cooling device.

28. The method according to claim 27, wherein the biological tissue is selected from the group consisting of parenchymal organ, blood-supplying vessel thereof, and combinations thereof.

29. The method according to claim 27, wherein the biological tissue is selected from the group consisting of gonad, a blood-supplying vessel thereof, and combinations thereof.

30. The method according to claim 27, wherein the biological tissue is selected from the group consisting of ovaries, testes, a blood-supplying and blood-draining vessel thereof, and combinations thereof.

31. The method according to claim 27, wherein the biological tissue is a biological tissue within the body of a patient to whom cancer therapy is applied during the method.

32. The method according to claim 27, wherein the heat absorption zone of the cooling unit is applied to the biological tissue within the body from outside the body in order to establish a heat-conducting contact between the heat absorption zone and the biological tissue within the body.

33. The method according to claim 32, wherein testes are cooled by placing the heat absorption zone on the testes from the outside, or ovaries are cooled by placing the heat absorption zone on an inner surface of the vagina.

34. The method according to claim 27, wherein the cooling device is arranged outside the biological tissue located within the body of a patient, wherein the thermal energy is released extracorporeally.

35. The method according to claim 27, wherein the hollow tube is guided extraperitoneally out of a patient's body by the shortest route.

36. The method according to claim 27, wherein a temperature of 8° C. to 14° C. is set at the heat absorption zone of the cooling unit.

37. The method according to claim 27, wherein the method is carried out by utilizing a device for in-situ cooling of biological tissue within the body, said device comprising
(i) a cooling device;
(ii) an implantable and sterile cooling unit having a heat absorption zone, wherein the heat absorption zone is suitable for establishing heat-conducting contact with a tissue within the body;
(iii) at least one hollow tube comprising or consisting of plastic, wherein the hollow tube connects the cooling device to the cooling unit; and
(iv) a substance for transporting thermal energy, wherein the substance for transporting thermal energy is arranged within the hollow tube.

38. The method according to claim 37, wherein the device comprises at least one further hollow tube comprising a plastic, wherein the at least one further hollow tube connects the cooling device to the cooling unit and the substance for transporting thermal energy is also arranged within the further hollow tube and wherein the at least one further hollow tube is connected to a second implantable and sterile cooling unit, wherein the second cooling unit is suitable for establishing a heat-conducting contact to a tissue within the body and has a second heat absorption zone and the device is configured to transport thermal energy from the second heat absorption zone of the second cooling unit to the cooling device.

39. The method according to claim 37, wherein the device comprises a skin port, which, reversibly or irreversibly, is connected to the at least one hollow tube, the cooling device and/or the cooling unit.

40. The method according to claim 37, wherein the device comprises a microcontroller which is configured to regulate the cooling device so that a temperature in the range from 8° C. to 14° C. prevails in the heat absorption zone of the cooling unit.

41. The method according to claim 37, wherein the device comprises a microcontroller which is configured to regulate the cooling device so that the substance for transporting thermal energy to the cooling device causes a cooling rate of 0.2 to 2.0 K/min in the heat absorption zone until a target temperature is reached in the heat absorption zone.

42. The method according to claim 37, wherein the device comprises a heating device, wherein the heating device
(i) has an implantable and sterile heating unit having a heat release zone suitable for establishing a heat-conducting contact with a tissue within the body;
(ii) has at least one hollow tube comprising or consisting of plastic, wherein the hollow tube connects the heating device to the heating unit; and
(iii) comprises a substance for transporting thermal energy from the heating device to the heat release zone of the heating unit, wherein the substance for transporting thermal energy is arranged within the hollow tube.

43. The method according to claim 37, wherein the cooling device is configured to apply, controlled by a microcontroller of the device, a cooling power to the heat absorption zone of the cooling unit, wherein the cooling power is calculated based on the formula
P [W]=3.7 [J/mL·K]·(310−x) [K]·Q [mL/sec]
wherein
x is the temperature in Kelvin to which cooling is to take place, and
Q is the volume flow in liters of blood per second in the biological tissue to be cooled.

44. The method according to claim 37, wherein the cooling device is configured, controlled by a microcontroller of the device, to
(i) increase its cooling power after starting the in-situ cooling of biological tissue within the body linearly up to a desired cooling power; and/or
(ii) drop its cooling power after stopping the in-situ cooling of biological tissue within the body linearly to a desired cooling power.

45. The method according to claim 37, wherein the cooling unit has a shape or consists of a shape which is selected from the group consisting of spiral, plate(s), dome, clip, clamp, half-shell, sleeve and combinations thereof.

46. The method according to claim 37, wherein the cooling unit and/or the at least one hollow tube has/have, at least in some regions,
(i) an outer surface that has a surface roughness Rz according to DIN EN ISO 4287 in the range from 50 to 800 μm; and/or
(ii) pores having a pore size of 50 μm to 1000 μm, measured with electron microscopy, wherein the pores comprise at least one antibiotic.
